(19) United States
(12) Patent Application Publication
(10) Pub. No.: US 2006/0229605 A1
(43) Pub. Date: Oct. 12, 2006

(54) Adjustable splint for osteosynthesis with incrementing assembly for adjustment in predetermined increments

(76) Inventor: Ron Anthon Olsen, Kingman, AZ (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/294,504

(22) Filed: Dec. 5, 2005

Related U.S. Application Data
(63) Continuation-in-part of application No. 11/083,566, filed on Mar. 18, 2005.
(63) Continuation-in-part of application No. 11/084,056, filed on Mar. 18, 2005.
(63) Continuation-in-part of application No. 11/083,547, filed on Mar. 18, 2005.

(51) Int. Cl. A61B 17/00

(52) U.S. Cl. 606/54

(57) ABSTRACT

Adjustable splints for treating bone breaks and fractures provide a variety of different options for adjusting the locations and configurations of the splints. Adjustable mounts on the splints can be moved to a variety of different locations with respect to the splint main body housings and bone connectors can be rotated into a variety of positions within the mounts. Main bodies of the splint devices can be conveniently moved with respect to each other into a variety of different configurations and positions. An incrementing assembly enables convenient movement of a mount in predetermined increments such that the practitioner or user can conveniently predict the amount of movement of the mount.

### CONTINUITY DATA

This patent application is a continuation-in-part of U.S. patent application Ser. No. 11/083,566, entitled “Adjustable Splint for Osteosynthesis with Modular Joint,” filed on Mar. 18, 2005, which is incorporated herein in its entirety by reference, and a continuation-in-part of U.S. patent application Ser. No. 11/084,056, entitled “Adjustable Splint for Osteosynthesis with Modular Components,” filed Mar. 18, 2005, which is incorporated herein in its entirety by reference, and a continuation-in-part of U.S. patent application Ser. No. 11/083,547 entitled “Adjustable Splint for Osteosynthesis,” filed on Mar. 18, 2005, which is incorporated herein by reference in its entirety.

### 1. THE FIELD OF THE INVENTION

This patent application is in the field of adjustable splints for osteosynthesis. More specifically, this application relates to an adjustable splint device useful for treating the fracture of bones, e.g., foot, ankle, wrist, hand, and facial bones.

### 2. BACKGROUND OF THE INVENTION

When fractured bones are properly splinted, they often are able to heal in an appropriate manner thereby simulating the shape and function of the previously uninjured, natural bone. Bone fixation devices are often employed in the treatment of fractures of small bones such as bones in the foot, hand, or maxiofacial regions, but also with a variety of different bone types. Such fixation devices are often known as minisplint devices, particularly when used in treatment of the small bones.

Typical minisplint devices feature a longitudinal support body and a pair of clamps mounted on the longitudinal support body. A clamp can be moved along the body through the use of an adjustable lead screw extending through the support body. Bone screws that are transverse to the longitudinal body connect to the clamps and secure the minisplint to the bone. By adjusting the lead screw, the position of the clamps can be moved with respect to the longitudinal support, thereby adjusting the size and configuration of the splint and the location of the transverse bone screws.

One limitation to typical adjustable minisplint devices is that the clamp connected to the longitudinal support is only moveable in an axial, linear direction with respect to the longitudinal support. The bone screws are also limited in their orientation. This dynamic limits the practitioner's options when attempting to set one or more bones using such minisplint devices.

Another limitation with typical devices relates to the positioning of one longitudinal support with respect to another longitudinal support. Such positioning typically results in limited movement, again reducing treatment options.

Yet another limitation associated with previous minisplint devices is that the lead screw used to provide adjustment of the bone clamps is retained in the longitudinal support body through the use of complicated multi-part systems that require a number of different parts to be added to the device assembly.

Another disadvantage of typical devices is that the lead screw of the devices projects outwardly from the elongated body, thereby exposing the lead screw to being inadvertently turned.

Another disadvantage of typical devices is that it can be difficult to determine how much the lead screw of the minisplint device is turning.

### BRIEF SUMMARY OF THE INVENTION

The adjustable splints of the present invention overcome the aforementioned disadvantages by providing a variety of different options for adjusting the locations and configurations of the splints. The adjustable mounts of the present invention can be moved to a variety of different locations with respect to the splint main body housings and the bone connectors can be rotated into a variety of positions within the mounts. Furthermore, the main bodies of the splint devices can be conveniently moved with respect to each other into a variety of different configurations and positions, thereby enabling them to be placed into a variety of different positions.

According to one embodiment, an adjustable splint for osteosynthesis comprises: (i) at least one main body; and (ii) first and second mounts coupled to the at least one main body, the first and second mounts adapted to couple to respective first and second bone connectors. One or more additional main bodies, e.g., two, three, four, five, etc., main bodies are also available, depending upon the required procedure.

In light of a unique slot design within the mounts, the bone connectors, e.g., bolts or screws may be moved from one position to another position (and a number of positions therebetween) within the mounts, thereby increasing the number of positions into which the splint may be placed.

To further increase the modularity and different positions of one embodiment of the splints of the present invention, at least one of the first and second mounts comprises: (i) an engaging member movably coupled to the at least one main body such that the engaging member is selectively moved from a first position to a second position with respect to the at least one main body; and (ii) a holding assembly movably coupled to the engaging member such that the position of the holding assembly can be adjusted with respect to the engaging member. The holding assembly can optionally be connected directly to one or more main bodies. The holding assemblies (whether connected directly to the mount or connected to an engaging member) can be rotated in a 360 degree range of motion, further increasing the number of positions available.

To enable the movement of the bone connectors with respect to the holding assembly, the holding assembly of one embodiment comprises: (i) a collar configured to grasp at least one bone connector; and (ii) a holder adapted to adjustably hold the collar therein.

The mounts may be connected in a variety of different manners to the main body or main bodies. For example, a lead screw can be positioned within a slot in a housing of the main body with a first end of the lead screw being rotatably coupled to a first end of the main body. The second end of the lead screw can be retained within the slot by a retaining member such as a split retaining ring positioned adjacent a terminal surface of the second end of the lead screw. The retaining ring retains the second end of the lead screw within the main body, thereby preventing the lead screw from extending out of the main body in an inconvenient fashion. The retaining ring can be mounted within a slot within the interior surface of the main body, for example.

In order to increase the different types of fractures that can be treated, the splint can have first, second, third, fourth or additional main bodies, each having respective mounts and bone connectors coupled thereto. In such a configuration, in order to increase the range of motion of the splint, the first main body can be coupled to the second main body such that the first main body can rotate in at least two different planes with respect to the second main body.

In one embodiment, one main body can move in three or more different planes with respect to another main body. This can be achieved, for example, through the use of a universal joint connecting the first main body to the second main body.

In yet another embodiment, a three part joint is employed, providing even further optional positions for the splints. In one such embodiment, three main bodies may be conveniently connected, each of which can be moved in at least two different planes with respect to each other, and the bone connectors of which can be moved in different planes, thereby enabling convenient fixation of complex multi-bone fractures.

The splints of the present invention are conveniently used for callous distraction, as bone reductive devices, and/or for dynamic compression of bones. The splints of the present invention may be conveniently used to treat fractures of the foot, hand, ankle, wrist, knee, or any other bone or joint.

The splints of the present invention can also be conveniently installed in one piece, or, optionally, in separate pieces, such as by first mounting a bone connector(s) with a mount coupled thereto onto a bone, then coupling a main body of the splint thereto. This may make installation in difficult places more convenient and is made possible because of the conveniently connectable components of the present invention.

In yet another embodiment of the present invention, an incrementing assembly causes the lead screw to turn in predetermined increments, so as to enable a practitioner to move the bone screws a desired, predetermined distance with respect to each other. This enables a practitioner to conveniently grow the bone.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an adjustable splint of the present invention, the arrows illustrating certain possible movement of the mounts, the bone connectors within the mounts, and the main bodies of the splint with respect to each other.

FIG. 2 illustrates an exploded perspective view of the splint of FIG. 1.

FIG. 3A illustrates a top view of a housing of the main body of the splint of FIG. 1, while FIG. 3B illustrates a side view of the main body comprising the housing and the lead screw therein.

FIGS. 4A-4C illustrate front perspective, side, and top cross sectional views, respectively, of an engaging member of the splint of FIG. 1.

FIGS. 5A-5C illustrate top, perspective, and cross sectional views, respectively, of a rear holder portion of the splint of FIG. 1.

FIGS. 6A-6C illustrate front perspective, rear perspective, and top views, respectively, of a front holder portion of the splint of FIG. 1.

FIGS. 7A-7D illustrate top, interior perspective, interior, and cross sectional views, respectively, of one part of a collar of the splint of FIG. 1, the opposing part of the collar having, in one embodiment, the same configuration.

FIG. 8 illustrates a mount assembly illustrating a perspective view of the arrangement of the mount components, including the collar and holder portions, with respect to each other.

FIGS. 9A-9C illustrate top, perspective, and cross-sectional views, respectively, of a rear holder portion of the splint of FIG. 1.

FIGS. 10A-10C illustrate top, side perspective, and side views, respectively, of a first joint portion of the splint of FIG. 1.

FIGS. 11A-11C illustrate top, side perspective, and side views, respectively, of a second joint portion of the splint of FIG. 1, the first portion configured to adjustably mate with the teeth of the second portion.

FIGS. 12A-12C illustrate the operation of the incrementing assembly that enables the lead screw to be turned in predetermined increments.

### DETAILED DESCRIPTION OF THE PREFERRED EMBODIMENTS

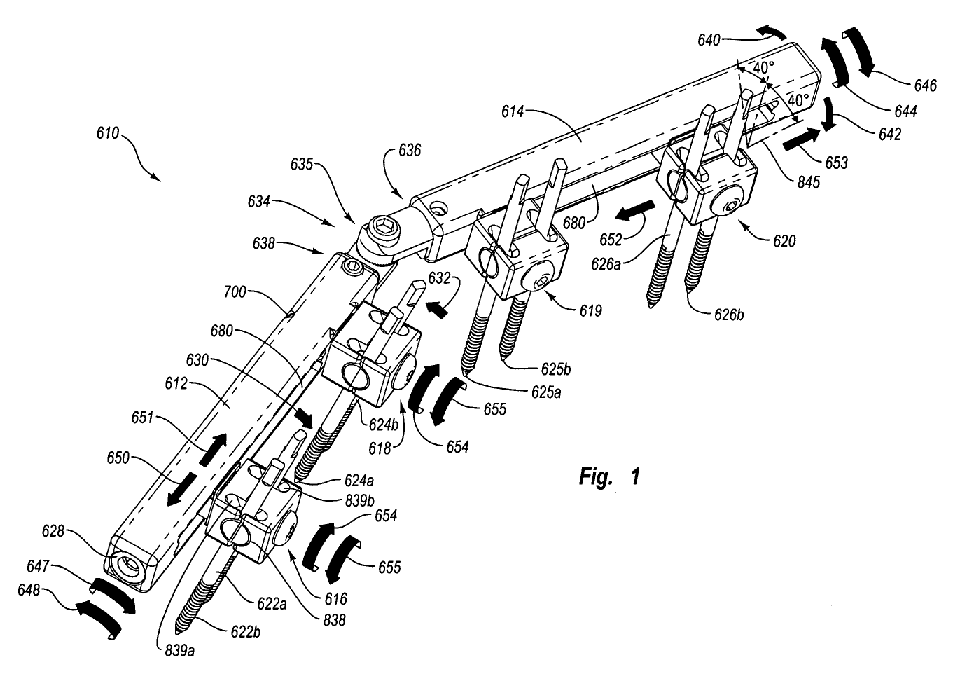

FIGS. 1-12C demonstrate examples of adjustable splints for osteosynthesis. FIG. 1 shows an adjustable splint 610 that is adjustable in more than one plane or axis, as illustrated by the arrows therein, thereby providing a variety of different, selectable splint configurations and treatment options for a practitioner. FIGS. 1-12C illustrate various individual components of splint 610.

With reference now to FIG. 1, splint 610 has first and second main bodies 612, 614 to which respective first, second, third, and fourth mounts 616, 618, 619 and 620 are coupled. Bone screws 622a-b, 624a-b, 625a-b, and 626a-b are coupled to respective mounts 616, 618, 619, 620, and connect splint 610 to one or more selected bones during a bone splinting procedure. Each of these components may be selectively moved with respect to each other in a variety of different advantageous configurations, as illustrated by the arrows shown in FIG. 1, which will now be discussed in additional detail.

One advantage of adjustable splint 610 is that bone connectors, e.g., bone screws 622a-b, 624a-b, 625a-b, 626a-b are movably coupled to respective mounts 616, 618, 619, 620 so as to selectively move from a first position within a respective mount 616, 618, 619, 620 to a second position within a respective mount, as reflected by arrows 630, 632. More specifically, whereas screws 626a-b are in a perpendicular position with respect to main body 614, arrows 630, 632 illustrate that, in contrast, bone screws 622a-b, and 624a-b have been moved away from a position that is perpendicular to main body 612. Thus, as shown in FIG. 1, screws 622a-b, 624a-b, 625a-b, and 626a-b are selectively rotatable from a first position to a second position within a respective mount 616, 618, 619, 620. The screws 622a-b, 624a-b, 625a-b, and 626a-b rotate about an axis transverse to a longitudinal axis of screws 622a-b, 624a-b, and 626a-b. Thus, first mount 616 adjustably receives bone screws 622a-b therein, such that screws 622a-b are selectively fixed in one of: (i) a first position within the mount; and (ii) a second position within the mount and are selectively moved from the first position to the second position within the mount. This enables a practitioner to position the bone screws into a position that is most useful for attaching splint 610 to one or more bones.

Joint 634 connects main body 612 to main body 614. Joint 634 is comprised of multiple components that have selectively moveable connections at three interfaces 635, 636, 638. Movement of main body 612 with respect to main body 614 can occur at each of the first, second, and third interfaces 635, 636, 638, respectively.

By moving at first interface 635, as shown in FIG. 1, joint 634 enables movement of second main body 614 with respect to first main body 612 in a first plane, as shown by arrows 640, 642. By moving at second interface 636, joint 634 enables movement of second main body 614 with respect to first main body 612 in a second plane, as shown by arrows 644, 646. Furthermore, by moving at third interface 638, joint 634 enables movement of second main body 614 with respect to first main body 612 in a third plane, as shown by arrows 647, 648.

FIG. 1 further illustrates that each of the mounts 616 and 620 are both slidable in the direction of arrows 650-651 and 652-653, respectively. Furthermore, FIG. 1 illustrates that mount 616 can be selectively rotated with respect to main body 612 in the direction of arrows 654 or 655. Mounts 618, 619, and 620 can also be similarly rotated with respect to main bodies 612, 614. In the embodiment shown, mounts 618, 619 are not slidable with respect to respective main bodies 612, 614 but rather are only rotatable with respect to respective main bodies 612, 614.

Thus, FIG. 1 illustrates various optional, advantageous configurations of splint 610, namely: (i) bone connectors 622a-b, 624a-b, 625a-b, and 626a-b can be moved back and forth within respective mounts 616, 618, 619, 620 (see arrows 630, 632); (ii) mounts 616, 618, 619, and 620 can be selectively rotated with respect to main bodies 612, 614 (see arrows 654, 655); (iii) mounts 616, 620 can be selectively moved longitudinally within respective main bodies 612, 614 (see arrows 650, 651 and 652, 653); (iv) main body 614 can be moved in a horizontal plane with respect to main body 612 (see arrows 640, 642); (v) main body 614 can be twisted along one axis with respect to main body 612 (see arrows 644, 646); and (vi) main body 614 can be twisted, along another axis with respect to main body 612 (see arrows 647, 648).

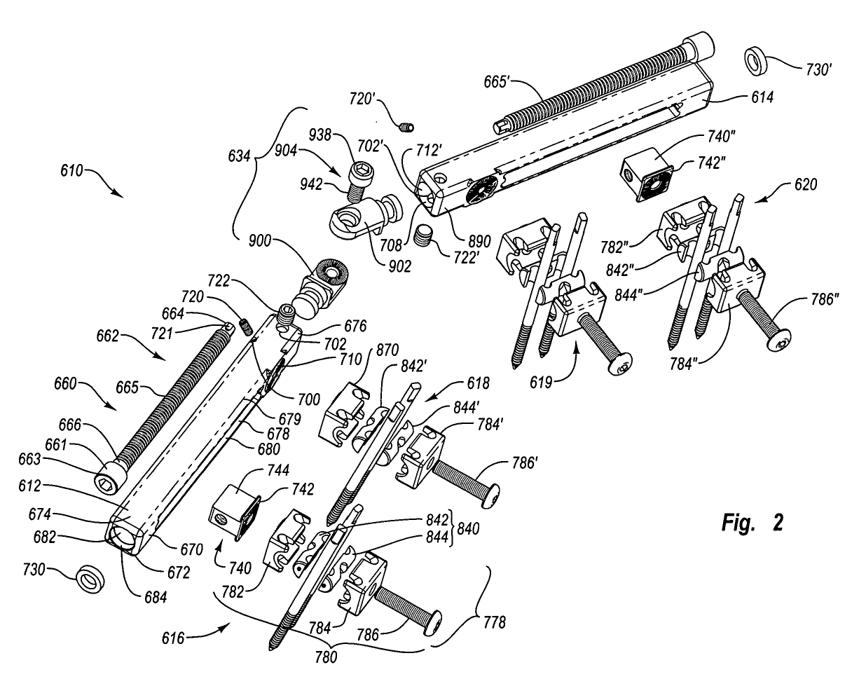

In order to describe the individual components of splint 610 in additional detail, an exploded view of splint 610 will now be discussed with reference to FIG. 2. FIG. 2 is an exploded, perspective view of the splint 610 shown in FIGS. 1 through 12C. As shown in FIG. 2, main body 612 comprises a lead screw 660 and a housing 670 configured to receive the lead screw 660 therein. Lead screw 660 is configured to rotatably mount within housing 670 of main body 612.

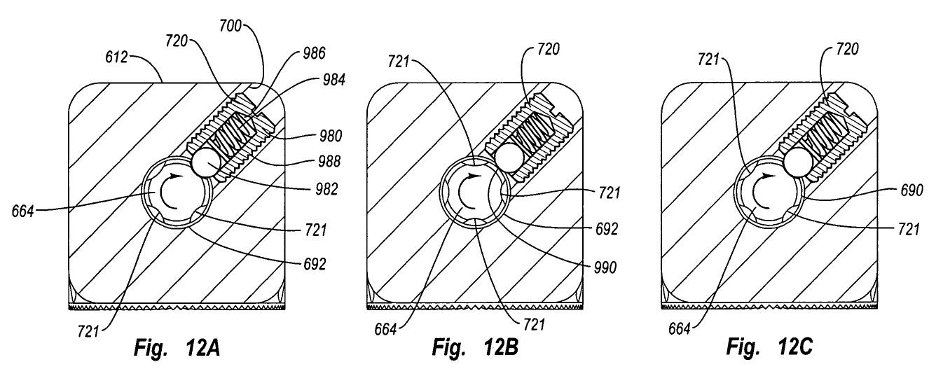

Lead screw 660 comprises an elongate screw having a proximal end 661 and a distal end 662. A head 663 is located at the proximal end 661 while a non-threaded tip 664 is located at the distal end 662. A threaded body 665 extends between the head 663 and the tip 664. Between head 663 and threaded body 665 is a smooth shoulder region 666. Head 663 is configured to receive a hex screwdriver tip therein in order to turn screw 660, although a variety of different shapes, configurations, and methods may be employed for turning the lead screw of the present invention. Tip 664 has a plurality of slots 721 therein that interact with a resistance member, as will be discussed in greater detail with respect to FIGS. 12A-12C.

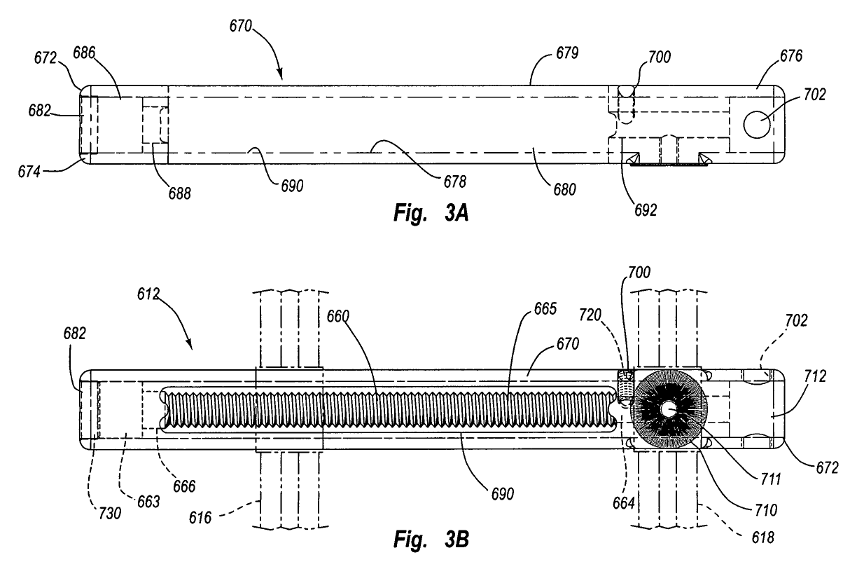

With reference now to FIGS. 2 and 3A-3B, housing 670, in which lead screw 660 is movably coupled, will now be described in additional detail. Housing 670 comprises an elongate, hollow body 672 having a proximal end 674, a distal end 676, an interior surface 678, and an exterior surface 679. Interior surface 678 defines a slot 680 in which elongate screw 660 is movably mounted. More specifically, interior surface 678 defines a proximal aperture 682 at proximal end 674, a head receiving chamber 686 (FIGS. 3A-B) adjacent proximal aperture 682, and an annular proximal internal shoulder 688 that is adjacent chamber 686. Proximal shoulder 688 has a diameter that is smaller than chamber 686. From annular proximal shoulder 688, slot 680 widens into slot body chamber 690, then narrows again at a distal annular internal shoulder 692. Adjacent distal annular internal shoulder 692 is a first top aperture 700 extending through body 672 of housing 670 to distal annular internal shoulder 692. First top aperture 700 receives a resistance member 720 that contacts tip 664 of screw 660 and provides resistance to the movement of screw 660 as it is turned. A second top aperture 702 also extends through body 672 of housing 670.

With reference now to FIG. 3B, which is a side view of main body 612, lead screw 660 is shown mounted within housing 670. As illustrated, lead screw 660 is placed in housing 670 by extending lead screw 660 through aperture 682 and inserting tip 664 of lead screw 660 into the portion of the slot defined by distal internal annular shoulder 692 (FIG. 3A), where tip 664 is retained within and rotates within the portion of the slot defined by shoulder 692. Threaded body 665 of lead screw 660 remains within slot body chamber 690 of housing 670 and shoulder region 666 of lead screw 660 is retained within and rotates within annular proximal internal shoulder 688. Head 663 of screw 660 is retained within and rotates within chamber 686.

As shown in FIGS. 2-3B, screw 660 is retained within slot 680 by a circular retaining member in the form of a ring 730 which is selectively placed within chamber 686 after screw 660 is placed within chamber 686, as shown in FIG. 3B. Ring 730 is configured to be pressed through aperture 682, then welded into chamber 686 during assembly, as reflected in FIG. 3B. Once lead screw 660 is placed within housing 670, retaining ring 730 is welded into chamber 686, thereby preventing lead screw 660 from exiting the interior surface of housing 670. This retains lead screw 660 within slot 680, as shown in FIG. 3B. In other words, ring 730 placed at the terminal end of screw 660 prevents screw 660 from extending proximally out of slot 680, while distal internal annual shoulder 692 prevents screw 660 from extending distally out of slot 680.

By rotating lead screw 660 within slot 680, mount 616, which is threadably coupled to lead screw 660, is selectively moved from one end of slot 680 to another end thereof, enabling a practitioner to achieve a configuration that is desirable for the setting of splint 610.

With continued reference to FIG. 3B, a circular array of teeth 710 is shown. A threaded slot 711 in the center of teeth 710 extends through body 672. Threaded slot 711 may extend through the entire body 672 or may extend only partially therethrough. As illustrated in FIGS. 2-3B, mount 618 is mounted on teeth 710. Also as shown, resistance member 720, is inserted into top aperture 700, thereby causing screw 660 to turn in predetermined increments with respect to main body 612. Resistance member 720 is configured to enable screw 660 to be turned in predetermined increments with respect to main body 612, as will be discussed with respect to FIGS. 12A-C.

By tightening screw 786′ (see FIG. 2), mount 618 is firmly, immovably positioned at a desired position with respect to housing 612. By loosening screw 786′, mount 618 is enabled to be rotated in a 360 degree range of motion in either direction with respect to housing 612. Screw 786′ may then be tightened again to couple mount 618 to a fixed position again.

Similarly, with continued reference to FIG. 3B, distal front hollow chamber 712 is shown. Distal front hollow chamber 712 has a circular cross section and is formed within body 672 and communicates with top aperture 702, which extends through body 672. As illustrated in FIGS. 2-3B, a first joint portion 900 of joint 634 can be inserted into distal front chamber 712 and a screw 722 or other connector is inserted through aperture 720 into distal front chamber 712, thereby retaining the cylindrical body of first joint portion 900 within distal front chamber 712. By tightening the screw 722, joint portion 900 is firmly, immovably positioned at a desired position within distal front hollow chamber 712. By loosening screw 722, joint portion 900 is enabled to be rotated in a 360 degree range of motion in either direction within chamber 712. An additional screw may also be placed in the bottom portion of second top aperture 702 to assist with retaining joint portion 900 therein.

Thus, threaded slot 711 (FIG. 3B) functions to facilitate the adjustable connection of mount 618 to main body 612, while aperture 702 and chamber 712 function to facilitate the adjustable connection of joint portion 900 to main body 612.

Mount 616 will now be discussed in additional detail. As shown in FIG. 2, mount 616 comprises: (i) an engaging member 740; and (ii) holding assembly 778 coupled to engaging member 740. The holding assembly 778 is comprised of a holder 780 and a collar 840 coupled thereto.

Engaging member 740 is movably (i.e., threadably) coupled to lead screw 660 of main body 612 and is selectively moved from a first position to a second position, with respect to main body 612, through the movement of lead screw 660. By turning lead screw 660, mount 616 slides back and forth, along the direction of arrows 650, 651 (FIG. 1). By employing the incrementing assembly of the present invention, mount 616 can be moved in predetermined increments with respect to main body 612, as discussed with regard to FIGS. 12A-C. This enables a practitioner or even a patient to move mount 616 as necessary in order to continue to grow bone, while healing from a surgical procedure, for example.

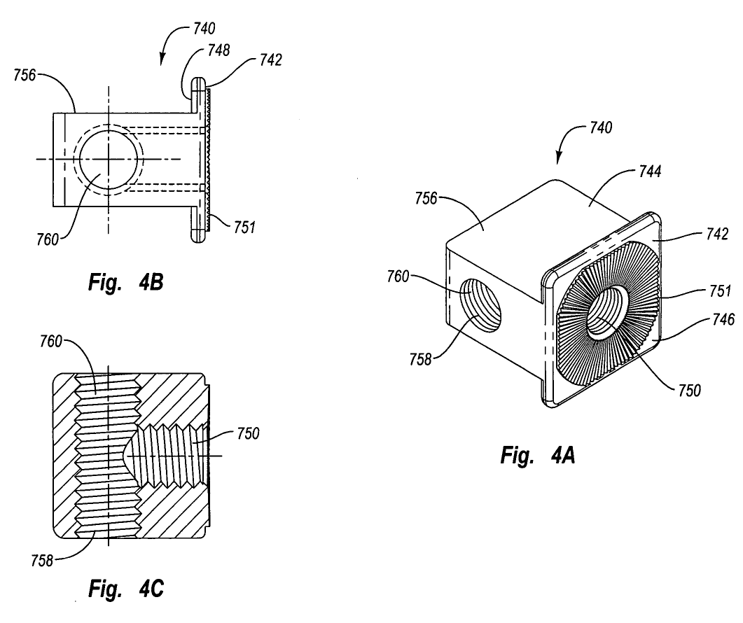

Engaging member 740 will now be described in additional detail, with reference to FIGS. 2 and 4A-4C. Engaging member 740 comprises a receiving member 742 and a sliding member 744, which, in the embodiment shown, extends integrally from receiving member 742.

Receiving member 742 has a front surface 746 and a rear surface 748 from which sliding member 744 extends. Receiving member 742 has a circular array of teeth 751, thereon. Extending through the circular array of teeth 751 is a threaded slot 750 that is configured to receive a bolt therein.

Extending from receiving member 742, e.g. in an integral fashion, is sliding member 744, which has an exterior surface 756 and an interior surface 758. The interior surface 758, defining a threaded slot 760, extends from one end of the sliding member 744 to the other. Thus, interior surface 758 defines a threaded slot 760 through which threaded body 665 of lead screw 660 threadably moves, thereby advancing sliding member 744 in a desired direction when sliding member 744 is placed within slot 680 of housing 670.

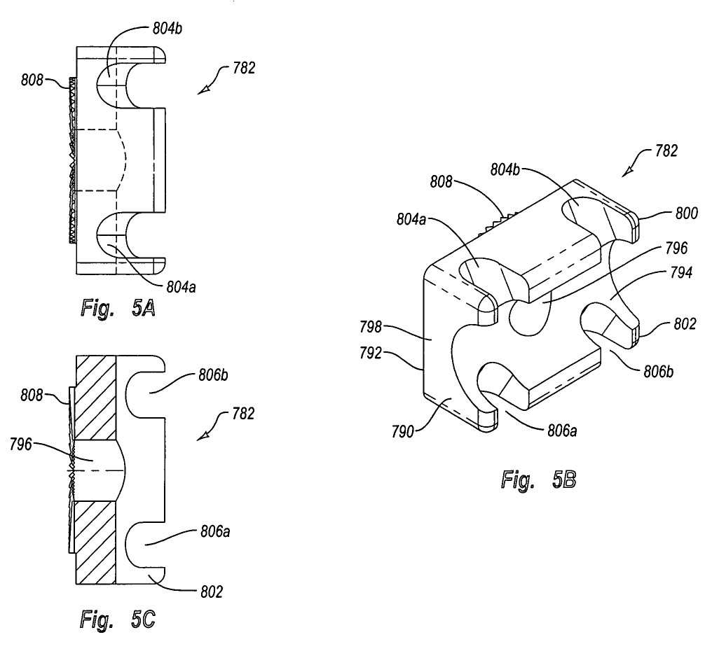

Engaging member 740, is also configured to adjustably connect to holding assembly 778, which holds bone connectors 622a-b. As mentioned, holding assembly 778 is comprised of a holder 780 and a collar 840 coupled thereto. Holder 780 holds collar 840 therein and comprises multiple components, which will now be described with continued reference to FIG. 2 and additionally to FIGS. 5A through 7D.

Holder 780 comprises a rear holder portion 782, a front holder portion 784, and a holding assembly connector, such as a bolt 786, which extends through front portion 784, through rear portion 782 and into threaded slot 750 of engaging member 740. The rear and front holder portions 782, 784 and bolt 786 that collectively form holder 780 are adapted to adjustably hold the two-piece collar 840. Bolt 786 thus extends through collar 840 to maintain collar 840 in a fixed position with respect to holder 780. Bolt 786 also extends into engaging member 740 to retain holding assembly 778 in a desired position with respect to engaging member 740. Bolt 786 can be tightened to maintain assembly in a desired position, then loosened to adjust the position of assembly 778.

Rear holder portion 782, will now be described in additional detail with reference to FIGS. 5A through 5C. Rear holder portion 782 comprises a U-shaped member 790, having an exterior surface 792 and a U-shaped interior surface 794. The interior surface 794 has a slot 796 therein. The U-shaped member has a central portion 798 and upper and lower leg members 800 and 802 extending therefrom, each of the leg members having corresponding groove portions 804a, 804b, and 806a, 806b, therein, respectively. In FIG. 5B, groove portion 804a is positioned above groove portion 806a, while groove portion 804b is positioned above groove portion 806b.

Coupled to the rear surface of U-shaped member 790 (e.g. integrally extending from U-shaped member 790) is a circular array of teeth 808. The slot 796 of U-shaped member 790 continues through teeth 808, as shown in FIG. 5C. Teeth 808 are configured to mate with teeth 751 (FIG. 4A) of engaging member

When holding assembly 778 is placed tightly against engaging member 740, teeth 808 of rear holder portion 782 engage teeth 751 of engaging member 740 such that rear holder portion 782 does not inadvertently rotate. Thus, when bolt 786 is threaded tightly through the remaining components of holding assembly 778 and into engaging member 740, holding assembly 778 does not inadvertently rotate. Thus, mating teeth 751 and 808 serve to prevent such inadvertent rotation, thereby ensuring that the bones are properly set. In order to rotate holder 780 in either direction in a 360 degree range of motion, bolt 786 is loosened, and mount 616 is then rotated in the direction of arrows 654 or 655 (FIG. 1), then bolt 786 is tightened again, thereby securing the position of holder 780 with respect to engaging member 740.

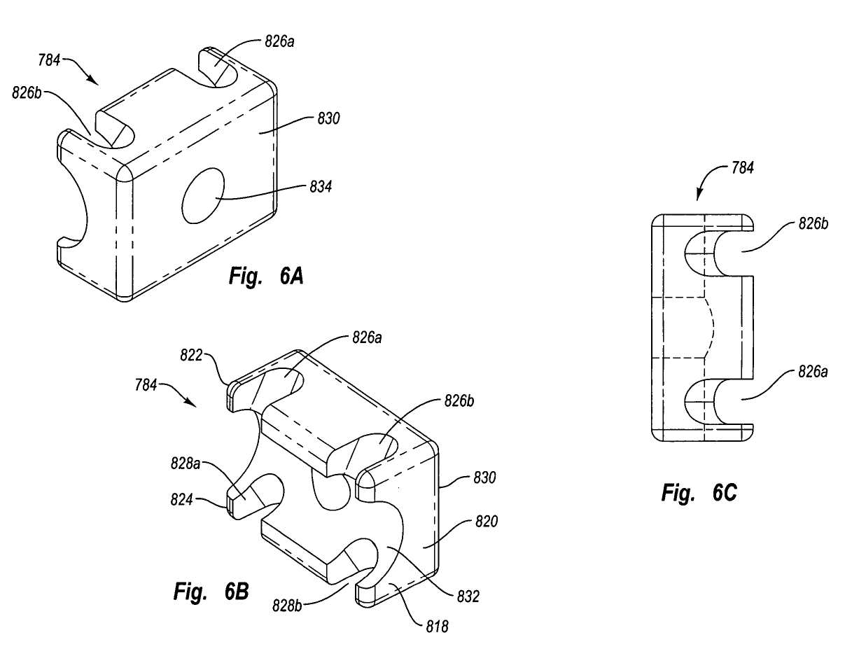

As mentioned above, in order to form holder 780, rear holder member 782 is combined through the use of a connecting member e.g., bolt 786, to front holder member 784, which will now be discussed with reference to FIG. 2 and FIGS. 6A through 6C. Similar to rear member 782, front holder member 784 comprises a U-shaped member 818 having a central portion 820 and upper and lower legs, 822, 824, extending therefrom, each leg 822, 824 having a pair of corresponding groove portions 826a, 826b and 828a, 828b therein, respectively. In FIG. 6B, groove portion 826a is positioned above groove portion 828a, while groove portion 826b is positioned above groove portion 828b.

Front holder member 784 has an exterior surface 830 and a U-shaped interior surface 832. A slot 834 extends from the exterior surface 830 to the interior surface 832, such that bolt 786 may be extended during assembly through front member 784 of holder 780 and through rear holder member 782 to thereby form holder 780.

As reflected in FIGS. 1 and 2, when front holder member 784 and rear holder member 782 are placed adjacent to each other, such that the U-shaped interior surfaces 832 and 794 are adjacent to each other in a symmetrical, complimentary fashion, a substantially circular cavity 838 (FIG. 1) is formed therebetween. Furthermore, the respective groove portions of rear holder member 782 and front holder member 784 combine to form elongate upper holder slots 839a-b (FIG. 1) and corresponding elongate lower holder slots which are formed below corresponding upper holder slots 839a-b. Upper holder slot 839a is formed from the combination of groove 804a of rear holder member 782 with groove 826b of front holder member 784. Upper holder slot 839b is formed from the combination of groove 804b of rear holder member 782 with groove 826a of front holder member 784. The lower holder slots are similarly formed from the combination of grooves 806a, 806b of rear holder member 782 with corresponding grooves 828b, 828a of front holder member 784. Each of the elongate upper holder slots 839a-b and the elongate lower holder slots extend from the exterior surface to the interior surface and communicate with the cavity 838.

The upper slots 839a-b and lower slots of holder 780 enable screws 622a-b to move in a range of motion, which in one embodiment is approximately 40 degrees in each direction (i.e. in the forward or backward direction), for a total 80 degree range of motion, as shown in FIG. 1 at 845. Two-part collar 840, which holds screws 622a-b within holder 780 and rotates within holder 780 in order to achieve such range of motion, will now be discussed in additional detail.

As shown further in FIGS. 1, 2, and 7A-7D, two-part collar 840 is a substantially cylindrically-shaped collar that fits within the substantially cylindrical-shaped cavity 838 that is formed between front holder member 784 and rear holder member 782. Collar 840 retains bone screws 622a-b within holder 780, i.e., rotatably within cavity 838 and also allows replacements of screws 622a-b so that differently sized and shaped screws 622a-b may be employed in splint 610.

As shown, collar 840 comprises a rear collar member 842 and a front collar member 844. Rear collar member 842 will now be discussed in additional detail, keeping in mind that in the embodiment of FIGS. 1 and 2, the front collar member 844 has the same or substantially similar configuration as that of rear collar member 842.

Rear collar member 842 comprises a substantially half-cylindrically shaped member, having an interior face surface 850 and an exterior rounded surface 852. Collar member 842 further has a first end portion 853a and a second end portion 853b and a central portion 855 therebetween. First and second opposing, substantially half-cylindrically-shaped parallel elongate grooves 854a-b are made in respective opposing ends 853a, 853b of the interior face surface 850, each extending from a top 855a of interior surface 850 to a bottom 855b thereof. Grooves 854a-b are perpendicular to the axis of rear collar member 842.

A third substantially half-cylindrically-shaped groove 856 is made in the exterior rounded surface 852 in the central portion 855 of collar member 842. Exterior rounded portion 852 has an exterior rim 869. An oval shaped slot 848 extends from interior rim 867 to exterior rim 869. Slot 848 thus extends from rim 867 of interior surface 850 to rim 869 of the exterior surface 852 in the central portion 855 of rear collar member 842.

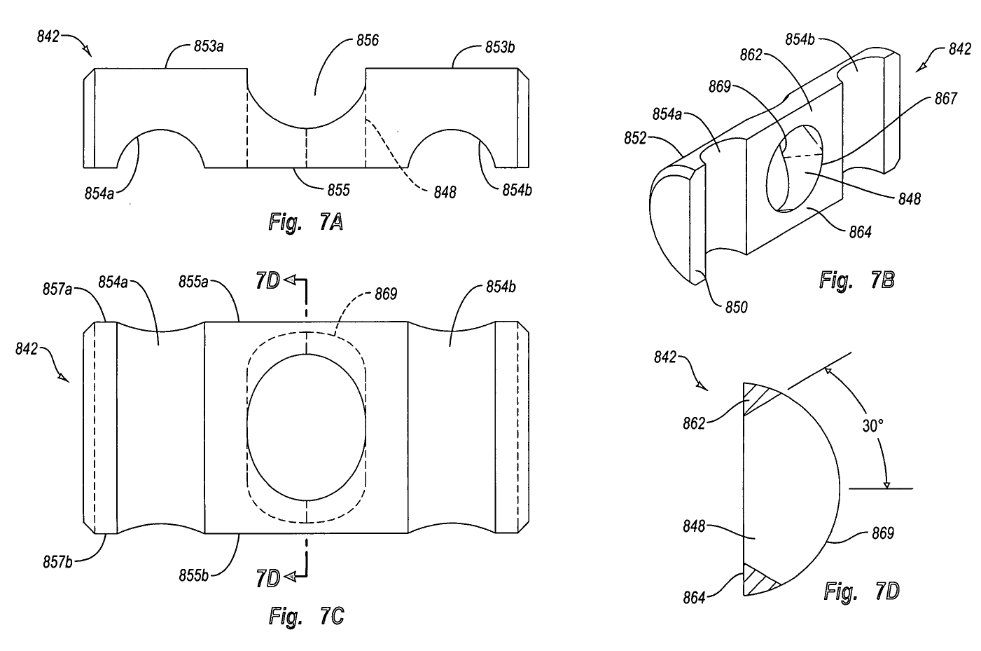

Slot 848 forms a passageway through which bolt 786 extends during assembly to properly orient collar 840 within holder 780. As shown, as slot 848 extends from interior surface 850 to exterior rounded surface 852, the size of the oval shaped slot 848 increases. Specifically, oval shaped slot 848 increases in size (i.e., top to bottom) as slot 848 extends from inner surface 850 to exterior rounded surface 852. This increase in size is an upward and downward flaring of slot 848 as it extends towards the exterior surface 852 and also reflects an increase in cavitation in the exterior rounded surface, as opposed to the inner surface 865. In one embodiment, slot 848 flares at an angle of approximately 30 degrees with respect to a longitudinal axis of slot 848, as shown in FIG. 7D.

This flaring and increased cavitation in the exterior rounded surface 852 enables each collar member 842, 844 to be rotated dramatically with respect to bolt 786, thereby enabling collar 840 to be rotated about bolt 786 when bolt 786 is extended through collar 840 and holder 780 once splint 610 is assembled.

Thus, the oval shaped aperture 867 is smaller in height than aperture 869, and as slot 848 extends from aperture 867 to aperture 869, the size of slot 848 increases. This increase in height enables the exterior rounded surface 852 of collar member 842 to be moved with respect to bolt 786 without significantly moving the interior surface 850 thereof.

As shown in FIG. 7D, in one embodiment, the angle of inclination of the upper surface (and in the embodiment shown, the lower surface) of slot 848 is about 30 degrees with respect to the longitudinal axis of slot 848. As a result, in one embodiment, collar 840 and the connectors retained therein can be moved about 40 degrees in each direction, for a total range of motion of about 80 degrees.

Grooves 854a-b correspond to similar or identical grooves of front collar member 844. Two-part collar 840 conveniently receives and retains screws 622a-b between corresponding grooves in collar members 842, 844. The collar 840 with screws 622a-b therein is placed within cavity 838 of holder 780, thereby enabling screws 622a-b to be retained within respective grooves 839a, 839b of holder 780 and to move into a desired orientation therein. Thus, in summary, screws 622a-b are held firmly between grooves 854a-b of collar member 842 and mating grooves of collar member 844. The screws move within the upper and lower slots of holder 780. Each collar member 842, 844 of the two-piece collar 840 has a slot 848 therethrough that is configured to receive a holding assembly connector 786 therethrough.

FIGS. 1 and 2 thus illustrate the combination of components that form mount 616. During assembly a user decides which screws 622a-b to use for a particular procedure, keeping in mind that a variety of different diameter screws 622a-b may be placed in collar 840. Upon selecting the desired screws (or optionally a single screw), collar members 842, 844 are mounted onto opposing sides of the screw(s) to form a screw/collar 840 assembly.

Rear and front holder members 782, 784 are then mounted onto respective opposing sides of the screw/collar 840 assembly such that screws 622a-b can be moved back and forth within upper holder slots 839a, 839b and lower holder slots. Attachment bolt 786 is then extended through holder member 784, collar member 844, collar member 842, and secured within holder member 782 such that holder members 782 and 784 are securely fixed to each other with collar 840 maintained tightly therebetween. When it is desired to adjust the position of collar 840, and hence screws 622a-b, attachment bolt 786 is loosened and collar 840 is rotated to a desired position. Attachment bolt 786 is then tightened, retaining collar 840 in the new desired position with respect to holder 780.

The holding assembly 778, which comprises collar 840 and holder 780 can be connected to engaging member 740 by mounting teeth 808 of rear holding member 782 on to mating teeth 751 of engaging member 740 and by placing bolt 786 through holding assembly 778 and threading bolt 786 into aperture 750 (FIG. 4A), thereby adjustably coupling assembly 778 to engaging member 740.

Hence, bone connectors 622a-b are housed within a substantially cylindrically shaped collar 840 that selectively rotates within a two-part holder 780 when bolt 786 is loosened. As mentioned, in one embodiment, collar 840 can rotate about 40 degrees in either direction, such that collar 840 can effectively rotate about 80 degrees. This approximately 80 degree; range of motion enables bone connectors 622a-b to be placed into a variety of different positions.

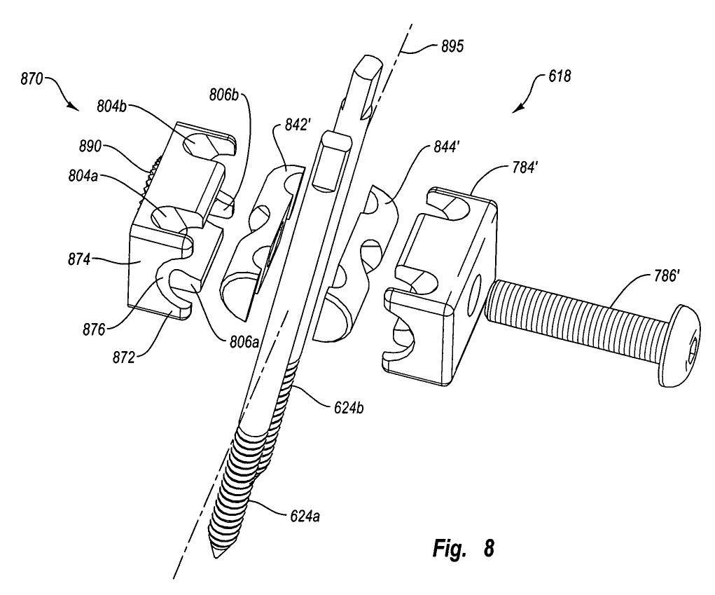

As further illustrated in FIG. 2, second mount 618 is also coupled to first main body 612. In the embodiment shown, mount 618 is not slidably coupled to first main body 612, but is rotatable in a 360 degree range of motion with respect to first main body 612, such that it can be adjusted into a desired position with respect thereto. Mount 618 will now be discussed in additional detail with respect to FIG. 8.

In the embodiment of FIG. 8, the components of mount 618 are the same or similar to those of mount 616, except that mount 618 does not have the engaging member 740 of mount 616. Thus, in one embodiment, the collar members 842′, 844′ of mount 618 are identical to respective collar members 842, 844 of mount 616 and the front holder member 784′ and bolt 786′ are identical to member 784 and bolt 786 of mount 616. However, in the embodiment shown, the rear holder member 870 of mount 618, shown in FIG. 8, is similar, but not identical to rear holder member 782 of mount 616, since the size of a portion of the rear holder member 870 is longer than that of member 782.

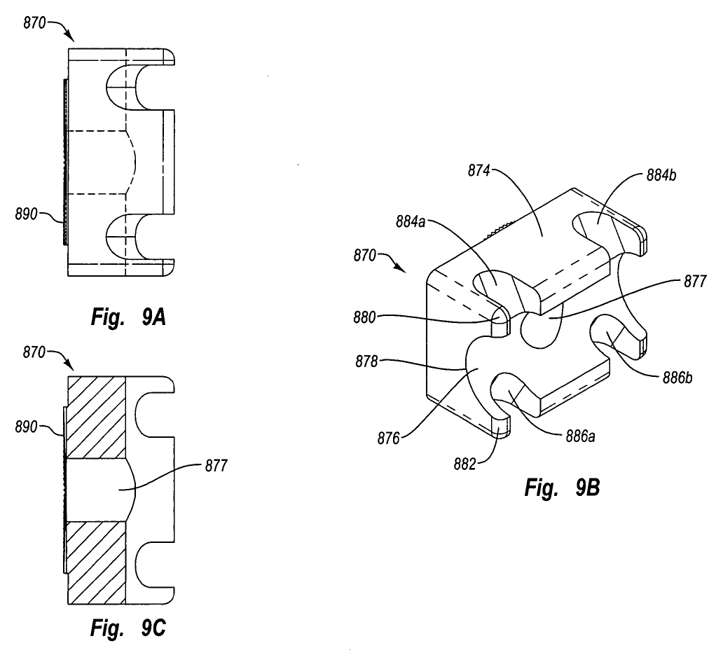

As shown in FIGS. 8 and 9A-C, similar to rear holder portion 782 of mount 616, rear holder portion 870 of mount 618 comprises a U-shaped member 872, having an exterior surface 874 and a U-shaped interior surface 876, which has a slot 877 therein, which is configured to threadably receive bolt 786′ therethrough. The U-shaped member 872 has a central portion 878 and upper and lower leg members 880 and 882 extending therefrom, each of the leg members having corresponding groove portions 884a, 884b, and 886a, 886b, therein, respectively.

Coupled to the U-shaped member 872 (e.g. integrally extending from U-shaped member 872) is a circular array of teeth 890. Teeth 890 are received in mating relationship with teeth 710 (FIG. 2) of main body 612 and bolt 786′ extends through portion 784′, portion 870, and collar portions 842′ and 844′ and into slot 711, thereby enabling convenient 360 degree rotation with respect to the main body 612.

The exterior surface 874 of rear member 870 is slightly longer than that of the exterior surface of member 782, such that rear member 870 extends to main body without the use of an engaging member, such as member 740. Optionally, however, an engaging member may be employed in another embodiment.

FIG. 8 also demonstrates an axis 895 that is transverse to the longitudinal axis of bone screw 624a. Bone screw 624a rotates about axis 895 when bone screw 624a is moved within mount 618. Axis 895 is also parallel to a longitudinal axis of main body 612. Thus, FIG. 8 illustrates that bone screw 624a is selectively movable about an axis 895 that is parallel to a longitudinal axis of main body 612.

FIGS. 1, 2 and 8 illustrate that the holder 780 substantially encloses the collar 840, thereby protecting the collar 840 and bone connectors received therein from the environment and providing an efficient, non-cumbersome mechanism which has relatively few moving parts. These figures illustrate that holder 780 encloses a first end of the collective collar 840 (corresponding to collar member end 853a in FIG. 7A), a second end of the collar 840 (corresponding to collar portion end 853b in FIG. 7A), and a central portion of the collar 840 located between the first and second ends of the collar 840.

With reference again to FIG. 2, main body 614 is also substantially similar to main body 612, although a variety of different designs may be employed. Main body 614 comprises a housing 890 that is similar to housing 670 of main body 612. Lead screw 665′ may operate similarly or identically to lead screw 665, for example and is retained in housing 890 through the use of ring 730′ welded into housing 890. A threaded spring-loaded plunger 720′ which is an example of a resistance member, is used to enable screw 665′ to turn in predetermined increments.

The mount 620 connected to main body 614 may have components that are similar or identical to the components of mount 616 connected to main body 612. Thus, mount 620 comprises: (i) an engaging member 740″ configured to selectively move along screw 665′; and (ii) a holding assembly coupled to engaging member 740″. The holding assembly comprises: (i) a holder comprising a rear holder member 782″, a front holder member 784″ and a bolt 786″configured to connect the holder assembly to engaging member 740″ front holder to the rear member; and (ii) a collar comprising first and second collar members 842″, 844″ configured to grasp first and second screws 626a-b therebetween and to selectively rotate within members 782″, 784″ when loosened and to be fixed therebetween when tightened. The holding assembly is connected to the engaging member 740″ through the use of one or more screws 786″ for example. These components and relationships of mount 620 may be identical to the description of the components of mount 616 described above, or may be similar thereto, for example. Mount 619 may have components that are similar or identical to mount 618, for example.

The joint 634 (FIG. 1) connecting main body 612 to main body 614 will now be described in additional detail with reference to FIGS. 1, 2 and 10A-10C. Joint 634 comprises a first joint member 900, a second joint member 902, and a connector, such as a bolt 904 or screw selectively connecting first joint member 900 to second joint member 902. Joint 634 conveniently acts as a universal joint, allowing movement in a variety of different directions.

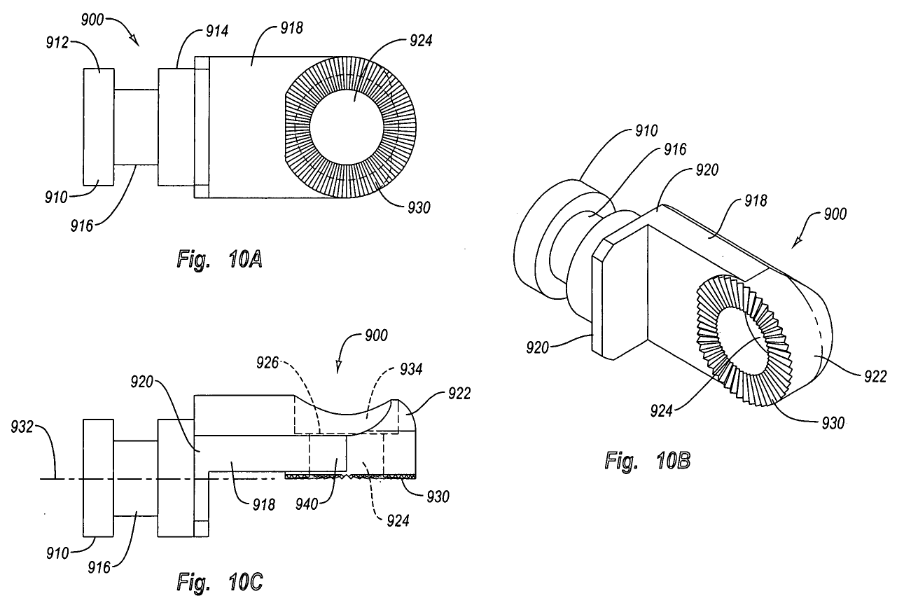

As illustrated in FIGS. 2 and 10A-C, first joint member 900 comprises: (i) a cylindrical body 910 having a first end 912 and a second end 914 and an annular groove 916 therebetween; (ii) an extension member 918 extending from the cylindrical body 910, the extension member 918 having a first end 920 that is coupled to the second end 914 of the cylindrical body 910 and a second end 922 having an aperture 924 therein; and (iii) a circular array of teeth 930 on the second end 922 of member 918 extending concentrically about the aperture 924 such that the aperture extends through the teeth 930 and the second end 922 of extension member 918. Cylindrical body 910 is received in mating relationship within chamber 712 of main body 612 and enables convenient 360 degree rotation with respect to the main body 612 selectively connected thereto. Extension member 918 extends away from cylindrical body 910 such that the longitudinal axis of extension member 918 is aligned with the longitudinal axis 932 (e.g., parallel to, or along the same axis) of cylindrical body 910. Hence, teeth 930 are oriented transversely to the longitudinal axis 932 of cylindrical body 910.

Aperture 924 defines a chamber 934 having an internal ridge 926 on which the head 938 of bolt 904 rests when joint 634 is assembled. Chamber 934 further comprises a passageway 940 through which the body 942 of bolt 904 extends during assembly.

Thus, in summary, first joint member 900 has (i) a cylindrical body 910 at a first end of joint member 900 which adjustably couples to first main body 612; and (ii) an aperture 924 at a second end of first joint member 900 about which teeth 930 extend.

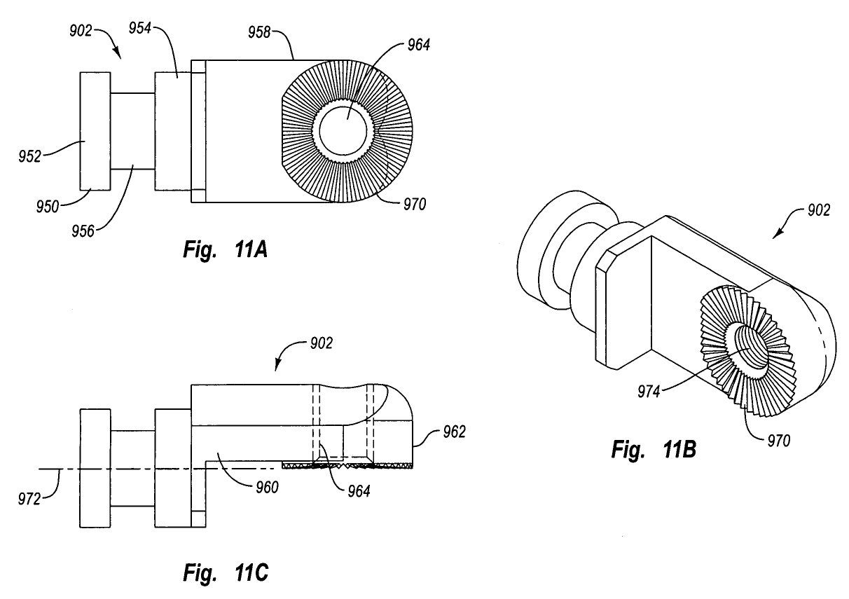

As illustrated in FIGS. 2 and 11A-C, second joint member 902 comprises: (i) a cylindrical body 950 having a first end 952 and a second end 954 and an annular groove 956 therebetween; (ii) an extension member 958 extending from the cylindrical body 950, the extension member 958 having a first end 960 that is coupled to the second end 954 of the cylindrical body 950 and a second end 962 having an aperture 964 therein; and (iii) a circular array of teeth 970 on the second end 962 of member 958 extending concentrically about the aperture 964, such that the aperture extends through the teeth 970 and the second end 962 of extension member 958. Cylindrical body 950 is received in mating relationship within chamber 712′ and enables convenient 360 degree rotation with respect to the main body 614 selectively connected thereto. Extension member 958 extends away from cylindrical body 950 such that the longitudinal axis of extension member 958 is aligned with the longitudinal axis 972 (e.g., parallel to, or along the same axis) of cylindrical body 950. Hence, teeth 970 are oriented transversely to the longitudinal axis 972 of cylindrical body 950. Aperture 964 defines a threaded passageway 974 through which the body 942 of bolt 904 extends during assembly. Second joint member 902 thus has (i) a cylindrical body 950 at a first end of joint member 902 which adjustably couples to second main body 614; and (ii) an aperture 964 at a second end of second joint member 902 about which teeth 970 extend.

Thus, during assembly, as illustrated in FIG. 2, the corresponding teeth of each joint member 900, 902 are aligned and placed adjacent each other in mating relationship, such that the apertures 924, 964 extending through each joint member 900, 902 are aligned so as to receive bolt 904 within both apertures 924, 964. Bolt 904 is then extended through aperture 924 of first joint member 900 and threaded into aperture 964 of second joint member 902, thereby retaining joint members 900, 902 in a fixed, aligned position with respect to each other. Upon desiring to adjust the orientation of one joint member with respect to the other, bolt 904 is loosened and the joint members 902, 904 are realigned (i.e., the teeth are realigned with respect to each other), after which the bolt 904 is replaced and tightened.

Each of the cylindrical bodies 910, 950 of respective joint members 900, 902 are selectively, adjustably connected to respective main bodies 612, 614 through the use of respective connectors such as screws 722, 722′ which are extended through respective apertures 702, 702′ when respective cylindrical bodies 910, 950 are placed in a desired position within respective front chambers 712, 712′. As shown, screw 722′ threads into lower aperture 702′ in the embodiment shown, while screw 722 threads into upper aperture 702. Upper and lower screws may be employed in each main body, or optionally only a single screw 722, 722′ may be employed for each such connection.

Thus, in order to adjust the orientation of main body 614 with respect to main body 612, bolt 904 (FIG. 2) may be loosened and main body 614 may be moved in the direction of arrow 640. Also, as mentioned above, in one embodiment, collar 840 and the connectors retained therein can be moved about 40 degrees in each direction. Such a range of motion is illustrated at mount 619 in FIG. 1, for a total range of motion, in one embodiment, of about 80 degrees for the collars described herein, such as collar 840, etc. Optionally, screw 722 of FIG. 2 (and/or a lower screw in main body 612) may be loosened and main body 614 may be twisted in the direction of arrow 648 of FIG. 1 or in the direction of arrow 647 of FIG. 1.

Through the use of joint members 900, 902, it is possible for each respective main body 612, 614 coupled thereto to achieve 360 degrees of rotation about a respective joint member 900, 902, i.e., about the cylindrical body thereof. Also, the use of interlocking teeth and the interlocking ridges and surfaces thereof enable the use of long lever arms and decrease the amount of potential displacement between joint members 900, 902.

In summary, as shown in FIG. 1, splint 610 is a highly adjustable and modular splint that can be used in a variety of different positions in order to treat a variety of different fractures or other breaks. Movement can occur in a variety of different planes and axes and from a variety of different positions to another. Thus, splint 610 is an example of a splint that has multi-faceted adjustability in a variety of different directions and positions. Adjustment can be achieved through sliding, rotating, twisting, back and forth movement, and in a variety of different manners, or in any combination thereof.

As shown in FIG. 2, both holder 780 and collar 840 are two-part assemblies. However, in another embodiment, the holder and collar, are each a single member rather than being two-part assemblies.

One embodiment of the present invention features at least one main body to which at least one adjustable mount is movably coupled. In yet another embodiment, two, three, four, five, etc. main bodies are employed.

Thus, although FIG. 1 illustrates first and second main bodies 610, connected to each other, in another embodiment, only a single main body, e.g., main body 610 is employed, such as by connecting mount 616 to one portion of a bone, while connecting mount 618 to another portion of the bone.

The cylindrical bodies of the joint members and/or rear holder portions disclosed herein have a variety of different advantages, such as enabling convenient, selective coupling to a main body and convenient rotation in a 360 degree range of motion about each such cylindrical body. For example, the mounts of the present invention may be coupled to the main body or bodies of a respective splint prior to installation. Optionally, however, one or more bone connectors with one or more respective mounts thereon may first be coupled to one or more bones, after which the main body or bodies can be connected to a respective mount or mounts. This may be useful in a setting in which it is difficult to place one or more bone connectors in a desired location. This is possible because of the convenient coupling of a mount to a main body, or of one main body to another main body, through the use of the aforementioned cylindrical bodies, which conveniently couple to respective main bodies, as shown. Thus, during installation, the mount(s) may be first coupled to a main body or bodies, or may be first coupled, along with one or more bone connectors, to a bone or bones, after which the main body is coupled to the mount(s).

The splints of the present invention are useful in a variety of different settings. For example, in one embodiment, the splints of the present invention can be used for callous distraction, e.g., the splint is first employed to compress two portions of bone with respect to each other, then after a period of time, such as a week, one or more mounts on a splint is moved away from one or more other mounts, thereby distracting the bone(s), causing the bone to grow. According to one procedure, the bone is first cut, then reattached and compressed for a period of time, then lengthened slightly on a regular basis to grow the bone.

As one option, the mounts on the splint can be adjusted often, e.g., by moving the mounts ¼ millimeter apart four times per day for a week, or other amount of adjustment as desired. A fracture can be thus reduced by first compressing, then gradually distracting portions of a bone. These regular adjustments can be performed by the practitioner or patient. In one embodiment, in order to achieve a desired thread ratio, one complete rotation of the lead screw is equal to one millimeter of translational movement of a mount along the axis of the screw.

Although mounts such as mounts 616, 618, and 620 are identified as possible mounts of the present invention, a variety of different mounts may be employed to connect a bone connector to a main body. Thus, a “mount” as referenced in this specification or the appended claims may be any material or structure that connects a bone connector to a splint main body.

Additional disclosure relating to the embodiments of the present invention is available in the U.S. patent applications filed on Mar. 18, 2005 and entitled “Adjustable Splint for Osteosynthesis with Modular Joint,” U.S. patent application Ser. No. 11/083,566 and “Adjustable Splint for Osteosynthesis with Modular Components,” U.S. patent application Ser. No. 11/083,547, and “Adjustable Splint for Osteosynthesis,” U.S. patent application Ser. No. 11/083,547, filed Mar. 18, 2005 each of which are incorporated herein by reference in their entirety.

With reference now to FIGS. 1-2 and 12A-12C, as mentioned above, in one embodiment, in order to achieve a desired thread ratio, one complete rotation of the lead screw is equal to one millimeter, or other predetermined increment, of translational movement of a mount, e.g., mount 616, along the axis of a screw 660. One manner in which this is achieved is through the use of an incrementing assembly that enables a practitioner to turn the lead screw 665 in desired increments.

One such incrementing assembly comprises: (i) a resistance member 720; and (ii) at least one slot 721 in screw 660 that is contacted by the resistance member 720 when screw 660 is moved. Multiple slots (e.g., 2, 3, 4, 5, 6, etc.) can be employed.

One example of such an incrementing assembly comprises a resistance member such as a spring-loaded ball plunger 720 that is coupled to housing 670, as illustrated in FIGS. 2 and 12A-C. Plunger 720 is threadedly coupled to housing 670 so as to contact tip 664 of screw 660. When a slot 721 of tip 664 is adjacent plunger 720, as shown in FIG. 12A, screw 660 is temporarily held in place by plunger 720. Thus, plunger 720 resists movement of screw 660 until a sufficient amount of twisting force overcomes such movement. Upon the application of sufficient twisting force, screw 660 can be turned, as shown by the arrow in FIGS. 12A-C, from the position of FIG. 12A until it contacts the adjacent slot 721, as shown in FIG. 12C. Upon the application of sufficient twisting force, screw 660 can then be turned until it contacts another slot 721, and so on.

Thus, as shown, springloaded plunger 720 causes lead screw 660 to turn in predetermined increments by contacting slots 721 in tip 664 of screw 660 as screw 660 is turned with respect to housing 670.

As shown in FIGS. 12A-12C, spring-loaded plunger 720 is placed within slot 700 in main body 612 so as to contact tip 664 of lead screw 660 as lead screw 660 is turned within housing 670. The movement of screw 660, as shown in FIGS. 12A-C, causes springloaded plunger ball 982 to snap within a particular slot 721 as screw 660 is turned with respect to plunger 720. Plunger 720 retains screw 660 in that position until screw 660 is turned again.

Slots 721 are placed a predetermined distance apart, such that when screw 660 is moved, it moves in predetermined increments, thereby causing the mount 616 to move along screw 660 in predetermined increments.

Since mount 616 moves in predetermined increments, e.g., 0.25 mm per quarter turn of screw 660, the practitioner, or even a patient can make necessary adjustments to splint 610 in order to “grow” the treated bone over time. This can be accomplished by moving first mount 616 with respect to second mount 618 in such predetermined increments, e.g., 0.25 mm per quarter turn of screw 660 according to a schedule selected by the practitioner, such as four times per day.

Spring-loaded plunger 720 comprises a hollow housing 980 having a ball 982 therein that is biased toward tip 664 of lead screw 665 by a spring 984 mounted within a chamber 986 of housing 980. Housing 980 has external threads 988 thereon such that it can be threaded into threaded slot 700. However, a variety of different types of resistance members can be employed in the present invention.

As shown in the continuum of FIGS. 12A-12C, as lead screw 665 is turned, slots 721 of screw tip 664 alternately contact ball 982. As the screw 660 is turned in the direction of the arrow in FIG. 12A, ball 982 is moved from a first slot 721 to the exterior surface 990 of screw tip 664 to a second slot 721.)

In one embodiment, slots 721 are evenly spaced about tip 664 such that movement from each slot to a neighboring slot represents a predetermined amount of translational movement of a mount, such as mount 616 along screw 660.

For example, when screw 665 is turned, as shown in FIGS. 12A-12C, such that ball 982 leaves one slot 721 and contacts a neighboring slot 721, mount 616 can be moved one quarter millimeter with respect to screw 660, for example. Thus, in one embodiment, a complete (i.e., 360 degree) rotation of screw 660 such that ball 982 leaves a particular slot 721, then is returned to that same slot 721, results in a movement of 1.0 mm of mount 616 with respect to screw 665.

In yet another embodiment of an incrementing assembly of the present invention, a resistance member such as a spring-loaded plunger is mounted on a lead screw and is turned with respect to one or more slots in the internal annular shoulder of the housing. In yet another embodiment of an incrementing assembly, the resistance member comprises a flexible tab, e.g., a flexible metal tab, that extends from housing 980 into a slot and clicks within the slots as the screw is turned.

In one embodiment of the present invention the threads of screw 660 are 10-32 threads, such that there are approximately 32 threads per inch. In one such embodiment, a quarter turn (i.e., ninety degrees) of the lead screw 660 results in a movement of mount 616 about 0.008 inch, i.e., about 0.2 mm. However, a variety of different predetermined increments are available such that the practitioner can know how far a mount 616 will move by turning the screw a desired number of turns. This will enable the practitioner to predictably adjust the distance between a pair of mounts, e.g., mounts 616, 618 by turning the screw a desired number of complete rotations or partial rotations.

In another embodiment, the threads of screw 660 are configured such that upon turning the lead screw 660 a quarter turn, the mount 616 moves about 0.25 mm. Thus, the mount 616 can move about 1 mm upon turning the screw 660 in a complete circle, i.e., by making four one-quarter turns.

According to one procedure, the bone of a patient is caused to grow 1 mm per day by turning the screw 660 a quarter-turn four times per day, e.g., once every six hours. Since it is so convenient to move the mount 616 by merely turning the screw 660, this procedure of growing the bone can be performed by the patient at home without the need for a physician to turn the screw 660.

In one embodiment, upon turning the screw 660, the practitioner will feel the spring-loaded plunger 720 being moved from one slot 721 to another, e.g., by feeling the screw driver, Allen wrench, or other tool conveniently move screw 660 a predetermined amount. Thus, the practioner's tactile sense is one form of indicia indicating the amount that a particular mount is being moved. In another embodiment, the practitioner will hear the spring-loaded plunger 720 contacting one slot 721, then another, e.g., by hearing a clicking noise. In yet another embodiment, the practitioner will see the screw driver, Allen wrench, or other tool, conveniently move a predetermined amount, e.g., a quarter turn. The practitioner also sees the mount 616 move a predetermined amount.

Thus, examples of various indicia of movement of a mount 616 a predetermined amount that are generated by the incrementing assembly of the present invention include: (i) the sight of a tool or mount 616 being moved in quarter turn increments, (ii) the clicking sound or other sound of the plunger 720 contacting neighboring slots 721, and (iii) the tactile feeling experienced by the practitioner when moving a tool while adjusting the screw 660. The indicia of movement may also be a combination of any of the foregoing indicia.

In one embodiment, indicia, such as millimeter indications, are provided on housing 670 adjacent mount 616 such that a practitioner can specifically, conveniently measure the amount that mount 616 has moved. Such indicia can be laser marked or tooled into housing 670 for example, or placed thereon through some other fashion.

One example of a spring-loaded plunger that can be employed in the present invention is a ball-nose spring plunger, as shown in FIGS. 12A-12C. Examples of such ball-nose spring plungers are commercially available from McMaster-Carr, P.O. Box 740100, Atlanta, Ga. 30374-0100, 6100 Fulton Industrial BLVD, Atlanta, Ga. 30336-2852. However a variety of different types of resistance members may be employed in the present invention.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An adjustable splint for osteosynthesis, comprising:
at least one main body having a first end and a second end;
first and second mounts coupled to the at least one main body, the first mount being movably coupled to the main body, such that the first mount moves in predetermined increments with respect to the main body, the first and second mounts being adapted to couple to first and second respective bone connectors.

2. A splint as recited in claim 1, wherein a screw moveably couples the first mount to the at least one main body.

3. A splint as recited in claim 2, wherein an incrementing assembly is configured to enable the screw to move in predetermined increments.

4. A splint as recited in claim 3, wherein the incrementing assembly comprises a resistance member configured to contact to screw and to cause the screw to turn in predetermined increments.

5. A splint as recited in claim 4, wherein the incrementing assembly further comprises at least one slot in the screw that is contacted by the resistance member when the resistance member is moved with respect to the main body.

6. A splint as recited in claim 4, wherein the resistance member comprises a springloaded plunger.

7. A splint as recited in claim 2, wherein the screw has a plurality of slots therein, the resistance member contacting the slots as the screw turns with respect to the main body.

8. A splint as recited in claim 7, wherein the slots are positioned in equal distances about the periphery of the screw.

9. A splint as recited in claim 7, wherein the distance between the slots is a predetermined distance.

10. A splint as recited in claim 2, wherein the movement of the screw with respect to the main body generates an indication of the movement of the screw.

11. A splint as recited in claim 10, wherein the indication of the movement of the screw is selected from the group consisting of (i) a tactile indication, (ii) a sound, (iii) a visual indication; and (iv) a combination thereof.

12. A splint as recited in claim 3, wherein the resistance member is coupled to the housing of the main body.

13. An adjustable splint for osteosynthesis, comprising:
at least one main body having a first end and a second end, the main body having a slot therein;
a screw having a first end and a second end, the screw being movably positioned within the slot; and
first and second mounts coupled to the at least one main body, the first mount being movably mounted on the screw, the first and second mounts being adapted to couple to first and second respective bone connectors, and
an incrementing assembly configured to enable the screw to move in predetermined increments with respect to the at least one main body.

14. A splint as recited in claim 13, wherein the incrementing assembly comprises a resistance member configured to contact the screw as the screw turns with respect to the at least one main body.

15. A splint as recited in claim 14, wherein the resistance member comprises a spring loaded ball plunger configured to contact the screw as the screw turns with respect to the main body.

16. A splint as recited in claim 14, wherein the screw contacts the resistance member as the screw moves with respect to the main body, the screw having at least one slot therein.

17. A splint as recited in claim 15, wherein the screw can be moved past the resistance member when a sufficient amount of force is applied to move the screw.

18. A splint as recited in claim 13, wherein the incrementing assembly comprises (i) a plurality of slots formed in the distal end of the screw and (ii) a spring loaded ball plunger configured to selectively bias into at least one of the slots and to move into another slot when the screw is turned, the movement of the ball plunger into an adjacent slot providing an indication that the screw is moved a predetermined length.

19. An adjustable splint for osteosynthesis, comprising:
at least one main body having a first end and a second end, the main body having a slot therein;
a screw having a first end and a second end, the screw being movably positioned within the slot; and
first and second mounts coupled to the at least one main body, the first mount being movably mounted on the screw, the first and second mounts being adapted to couple to first and second respective bone connectors, wherein the screw is configured to move in predetermined increments with respect to the at least one main body.

20. A splint as recited in claim 19, wherein indicia indicate indicate that the screw is being moved with respect to the main body, the indicia selected from the group consisting of sound, tactile indicia, and visual indicia.

21. An adjustable splint for osteosynthesis, comprising:
at least one main body having first end and a second end, the main body having a slot therein;
a screw having a first end and a second end, the screw being movably positioned within the slot, the first end of the screw being movably coupled to a first end of the main body;
first and second mounts coupled to the at least one main body, the first mount being movably mounted on the screw, the first and second mounts being adapted to couple to first and second respective bone connectors; and
a resistance member coupled to the main body so as to contact the screw as the screw moves with respect to the main body, the resistance member causing the screw to move in predetermined increments with respect to the main body such that the first mount moves in predetermined increments with respect to the main body.

22. An adjustable splint as recited in claim 21, wherein the resistance member comprises a spring plunger biased into a slot in a distal end of the screw.

23. An adjustable splint as recited in claim 21, wherein the screw has a plurality of slots therein and wherein a member is biased into at least one slot in the screw, wherein movement of the screw causes the member to move from one slot to the next slot, thereby causing the screw to move in predetermined, measurable increments, such that the first mount is moved a desired, predetermined distance each time the screw is moved from one position to the next position.

24. A splint as recited in claim 21, wherein the screw moves from a first position to a second position, the first position being represented by the resistance member being disposed within one slot and the second position being represented by the resistance member being disposed within a second slot.
